(19) United States
(12) Patent Application Publication
(10) Pub. No.: US 2004/0087606 A1
(43) Pub. Date: May 6, 2004

(54) Shivering suppression during cooling to lower body temperature

(76) Inventors: Marc E. Voorhees, Arvada, CO (US); Richard M. Zweifler, Mobile, AL (US)

Correspondence Address: MARSH, FISCHMANN & BREYFOGLE LLP, 3151 SOUTH VAUGHN WAY, SUITE 411, AURORA, CO, 80014, US

(21) Appl. No.: 10/405,301

(22) Filed: Apr. 2, 2003

Related U.S. Application Data
(60) Provisional application No. 60/423,106, filed on Nov. 1, 2002.

(51) Int. Cl. A61K 31/485; A61K 31/445; A61K 31/4178; A61K 31/403

(52) U.S. Cl. 514/282; 514/317; 514/411; 514/397

(57) ABSTRACT

Active cooling of a person, such as to induce mild/moderate hypothermia, is accomplished by transferring heat from the persons body. Heat transfer and patient comfort are aided by administration of an anti-shivering drug and an anti-emetic drug.

### CROSS-REFERENCE TO RELATED APPLICATIONS

This application claims priority under 35 U.S.C. § 119 to prior U.S. Provisional Patent Application No. 60/423,106, filed Nov. 1, 2002, entitled “SHIVERING SUPPRESSION DURING COOLING TO LOWER BODY TEMPERATURE”, the entire contents of which are incorporated herein as if set forth herein in full.

### FIELD OF THE INVENTION

The invention relates to active cooling of the human body for therapeutic purposes, such as to induce hypothermia in people suffering a condition involving reduced blood supply.

### BACKGROUND OF THE INVENTION

Therapeutic induced mild-moderate hypothermia can be beneficial for people suffering stroke, myocardial infarction, cardiac arrest and other conditions involving reduced blood supply. One method for lowering body temperature is to insert a cooling device into an artery of the patient and to internally cool the patient's body by introducing a cooling fluid into the device. One concern for such endovascular techniques is the invasive nature of the procedure. A non-invasive technique for lowering body temperature is to externally cool the surface of the patient's body. Such exterior surface cooling could be achieved by directing the flow of a cooling fluid, such as cool air, around the patient's body. A more preferred external surface cooling technique is to apply a contact-cooling pad to the exterior surface of the patient and to circulate a cooling fluid, such as water or an aqueous solution, through the contact pad to cool the patient.

As a patient's body is cooled, shivering is likely to occur as a thermoregulatory response to the cooling. Shivering can increase heat production as much as 600% above basal levels. When shivering occurs during inducement of hypothermia, the additional heat production counteracts the active cooling of the patient's body and can significantly delay or even prevent attainment the desired lower body temperature.

An anti-shivering drug can be administered to the patient to suppress shivering during therapeutic inducement of hypothermia. Meperidine is one drug used for treatment to suppress post-operative shivering. Because conditions during induced hypothermia are much more severe than in the post-operative situation, the use of meperidine for shivering suppression during induced hypothermia may require administration of larger quantities of the drug for temperature management. With administration of large doses of meperidine, there is a concern for possible respiratory depression and nausea and vomiting. Even when not creating a medical concern, it would be desirable to suppress nausea and vomiting for patient comfort. Also, it is normally desirable to maintain the patient as conscious and responsive as possible, to facilitate monitoring of the patient's condition, such as for indications of neural impairment.

### SUMMARY OF THE INVENTION

With the invention, an anti-emetic drug may be administered in combination with administration of an anti-shivering drug for shivering suppression. The use of the anti-emetic drug may permit a level of shivering suppression to be attained with administration of a reduced quantity of the anti-shivering drug relative to use of the anti-shivering drug alone, and with reduced incidence of nausea and vomiting.

In one implementation, the invention may involve a method for lowering body temperature of a human patient, with the method involving transferring heat from the patient's body to cool the patient's body, administering an anti-emetic drug to the patient and administering an anti-shivering drug to the patient. The anti-emetic drug and the anti-shivering drug are administered in therapeutically effective quantities respectively for suppression during heat transfer of nausea and vomiting and of shivering. The lowering of the body temperature may be for any purpose, such as for example to reduce body temperature from an elevated level to normal (normothermia) in the case of fever or to reduce the body temperature to below normal (hypothermia) in the case of stroke, myocardial infarction or cardiac arrest.

The anti-shivering drug may comprise one or more substance effective for suppressing shivering. A variety of such anti-shivering drugs are known or may be identified in the future. Examples of some reported anti-shivering drugs include certain non-opioid analgesics (for example, tramodol and nefopan), certain opioid analgesics (for example, alfentanil, morphine, fentanyl, meperidine, naloxone and nalbuphine), certain α₂-andrenergic agonists (for example, clonidine and dexmedetomidine) and certain serotonin antagonists (for example, ketanserin and ondansetron). Also, multiple anti-shivering drugs may be used to the extent they are pharmacologically compatible. Moreover, it should be appreciated that drugs are often administered in the form of pharmacologically acceptable salts, so for example, the anti-shivering drug may be such a salt of any of the foregoing listed compounds. Meperidine, or a salt thereof, is particularly preferred for use as the anti-shivering drug. Unless otherwise required by the specific context, when a reference is made herein, including in the claims, to a drug compound, the reference includes the named compound and chemical variations of the named compound, and particularly includes salts of the compound, that are pharmacologically acceptable for administration to human subjects.

The anti-emetic drug is different than the anti-shivering drug, and the anti-emetic drug may comprise one or more substance effective for suppressing nausea and/or vomiting. A variety of such anti-shivering drugs are known or may be identified in the future. Examples of some anti-emetic drugs include certain D₂ dopamine antagonists/blocking agents (for example, phenothiazine antipsycotics such as prochlorperazine, triflupromazine, chlorpromazine and trifluorperazine; and metoclopramide), promethazine (both a D₂ dopamine antagonist and an H₁ histamine antagonist), certain antihistamines with anticholinergic effects (for example, diphenhydramine, dimenhydrinate and meclizine), and certain serotonin antagonists (for example, ondansetron, granisetron and tropisetron, which are 5-HT₃ antagonists). Preferred for use as the anti-emetic drug is one or more 5-HT₃ antagonist. Also, multiple anti-emetic drugs may be used to the extent they are pharmacologically compatible. Moreover, as noted above, drugs are often administered in the form of pharmacologically acceptable salts, so for example, the anti-emetic drug may be such a salt of any of the foregoing listed compounds. Ondansetron, or a salt thereof, is particularly preferred for use as the anti-emetic drug.

In one preferred implementation of the invention, the anti-emetic drug comprises a 5-HT₃ antagonist and the anti-shivering drug comprises an opioid analgesic. A particularly preferred pairing is for the anti-emetic drug to comprise ondansetron, or a salt thereof, and the anti-shivering drug to comprise meperidine, or a salt thereof. In one particularly preferred implementation, the use of the anti-emetic drug in combination with the anti-shivering drug reduces the quantity of the anti-shivering drug required to treat for the shivering suppression, relative to use of the anti-shivering drug alone. For example, an anti-emetic drug can be selected that independently acts to provide at least some level of shivering suppression. This is the case, for example, with the use of ondansetron, which as noted above has been reported for shivering suppression.

As noted above, each of the anti-emetic drug and the anti-shivering drug should be administered in therapeutically effective quantities. By “effective quantity” or “therapeutically effective quantity” of the anti-emetic drug and the anti-shivering drug, it is meant that the drug at issue is administered at a dose and with a dosing regimen that is pharmacologically acceptable to treat for the target condition under the circumstances. As will be appreciated, different people will have varying responses to an “effective quantity”.

### DETAILED DESCRIPTION OF PREFERRED EMBODIMENTS

To provide the suppression of shivering while the patient's body temperature is being lowered or while the temperature is in a lowered state, at least a portion of the anti-emetic drug and at least a portion of the anti-shivering drug should be administered prior to commencing the transfer of heat from the body to cool the body and/or while the transfer of heat from the body is being performed during cooling of the body. The method of administration may be by any suitable administration technique, such as, for example continuous infusion or oral administration. One convenient technique is by intravenous injection as needed.

The anti-emetic drug and the anti-shivering drug may each be administered separately. Each of the anti-emetic drug and the anti-shivering drug may be administered as a single dose, or using a multiple dose regimen involving an initial dose followed by one or more successive, and usually smaller, doses. For the anti-shivering drug, a multiple dose regimen may involve an initial dose to commence the shivering suppression followed by successive doses being administered as required to maintain the shivering suppression. The timing for subsequent dose administrations may be determined, for example by visual observation of the patient for indications that an onset of shivering is approaching. For the anti-emetic drug, a single dose given prior to or soon after commencement of active cooling may be sufficient, although a multiple dose regimen may be better when it is anticipated that cooling and body temperature suppression may extend for a significant duration. For example, an initial dose of the anti-emetic drug may be followed later by one or more supplemental doses as appropriate based on the pharmacokinetic properties of the specific anti-emetic drug. In one preferred implementation, at least a portion of the anti-emetic drug is administered to the patient prior to commencement of the cooling heat transfer, and also preferably prior to administration of any of the anti-shivering drug.

The active cooling of the patient's body to lower the temperature of the body may be accomplished by transferring heat from the patient's body. This heat transfer may be accomplished using any cooling equipment and techniques, or any combination of such equipment and techniques, including for example, the use of forced air cooling, contact cooling pads and/or the use of endovascular cooling devices and techniques.

In one implementation, the heat transfer may be accomplished with the use of a heat transfer fluid that is at a temperature that is lower than the body temperature of the patient. Air has a low heat capacity, and it is more difficult to achieve quick lowering of the body temperature using air or other gases as a heat transfer fluid, such as with forced air cooling. Liquids generally have higher heat capacities and are preferred for use as a heat transfer fluid.

To effect the heat transfer to cool the patient's body, the heat transfer fluid may be brought into heat transfer communication with the patient's body. With forced air cooling this could be accomplished for example by forcing the air to flow past at least a portion of the body to directly contact the body. A heat transfer liquid could also be directly contacted with a patient's body to effect a transfer of heat from the body, such as for example, by immersing a portion of the patient's body in a reservoir of the liquid.

Heat may also be transferred from the patient's body to a heat transfer fluid without direct contact between the fluid and the body. For example, the fluid may be contained within a heat transfer device that contacts a portion of the body. For example, when using an catheter device located within the vaculature of the patient, the heat transfer liquid may be forced to flow through the device, with heat being conducted from the patient across one or more walls of the device and into the heat transfer fluid, which can be removed from the device to remove the heat from the vicinity of the patient, effecting the desired cooling.

In one preferred implementation, the heat transfer device is contacted with an external dermal surface of the patient. Establishing good heat transfer communication may be aided, for example, by the use of a gel, ointment or other medium between the heat transfer device and the dermal surface. A heat transfer fluid, preferably a liquid, may be circulated through the heat transfer device, wherein heat is conducted from the dermal surface, across one or more wall of the heat transfer device and into the heat transfer fluid.

A contact pad is one preferred heat transfer device for cooling by contact with a dermal surface. Preferably, the contact pad includes an adherent surface to achieve intimate contact with a patient's skin. Such surface may be provided by a thermally-conductive, hydrogel layer juxtaposed to a fluid circulation layer. In the latter regard, the fluid circulation layer may comprise fluid channels and dimples disposed to modify fluid flow characteristics for enhanced heat transfer performance. Examples of contact pads and their operation are disclosed in U.S. Pat. No. 6,197,045 entitled “COOLING/HEATING PAD AND SYSTEM”, U.S. Pat. No. 6,375,674 entitled “COOLING/HEATING PAD AND SYSTEM”, and U.S. patent application Ser. No. 10/087,533 having a filing date of Feb. 27, 2002, entitled “IMPROVED MEDICAL THERMAL ENERGY EXCHANGE PAD”, the entire contents of each of which are incorporated by reference herein as if set forth herein in full. One example of a heat transfer system using one or more contact pad(s) is described below.

The system includes at least one heat exchanger to effect cooling of a fluid and a circulating pump for circulating the fluid through the heat exchanger and one or more interconnectable contact pad(s). Preferably, circulated fluid is drawn through the contact pad(s) under negative pressure. Such negative pressure may be established by locating the circulating pump downstream of the contact pad(s), wherein fluid is effectively pumped out of the contact pad(s) and then through the heat exchanger and back into the contact pad(s).

One or a plurality of fluid reservoirs may be located downstream of the heat exchanger. For example, a first fluid reservoir may be utilized to contain fluid that is removable therefrom to initially fill and then circulate through the contact pad(s). During normal heat exchange operations, fluid is circulatable through the contact pad(s) and the heat exchanger by the circulating pump substantially free from passage through the first fluid reservoir. The system may also comprise a second fluid reservoir through which fluid is circulated during normal heat exchange operations. By way of example, the first reservoir may be located to provide direct gravity fluid flow to the second reservoir. Further, the first reservoir may be vented to facilitate gas removal from the system.

In addition to the noted features, the system may further include various sensors to provide user feedback and automated control functionalities, thereby yielding enhanced patient thermal regulation. For example, the system may include a pressure sensor fluidly interconnected between an inlet side of the circulating pump and an outlet port of the interconnectable contact pad(s). Such pressure sensor may provide an output pressure signal employable (e.g. by a processor) to control the circulating pump (e.g. the flow rate therethrough) and thereby maintain negative pressure within the contact pad(s) within a predetermined range (e.g. thereby maintaining the desired flow rate through the contact pad(s)). Further, user alerts may be provided when the measured fluid pressure is within a predetermined range.

One or more fluid temperature sensors may also be utilized for sensing the temperature of the circulated fluid and providing an output temperature signal(s) in response thereto. The output temperature signal(s) may be utilized (e.g. by a processor) to control the operation of the heat exchanger. For example, the fluid output temperature signal(s) may be utilized to adjust the fluid temperature to within a predetermined range (e.g. as preset by a user). In this regard, patient temperature may also be monitored, wherein a patient temperature signal and the sensor output temperature signal(s) may be employed in combination to adjust the circulated fluid temperature. As may be appreciated, such temperature control functionality may be advantageously employed with anti-shivering and anti-emetic drugs in accordance with present invention.

The above-noted system features and additional system functionalities may be incorporated as disclosed in U.S. patent application Ser. No. 09/976,197, having a filing date of Oct. 11, 2001, entitled “PATIENT TEMPERATURE CONTROL SYSTEM WITH FLUID TEMPERATURE RESPONSE”, and U.S. patent application Ser. No. 10/233,843, having a filing date Sep. 3, 2002, entitled “PATIENT TEMPERATURE CONTROL SYSTEM WITH FLUID TEMPERATURE RESPONSE”, the entire contents of each of which are hereby incorporated by reference as if set forth in full hereinbelow.

The degree by which a patient's body temperature is to be lowered will affect the amount of cooling that is used. In some implementations, the patient's body will be lowered by at least 0.5° C., in other implementations by at least 1° C. and by yet other implementations by at least 2° C. In most situations, the body temperature will be lowered by no more than 5° C. When reference is made to a person's body temperature, the reference is to the core temperature of the person, not the temperature of extremities. An indication of the core temperature is determinable, for example by measuring the temperature at a person's tympanic membrane, in a person's rectum or in a person's bladder. The degree by which a person's core temperature is lowered is determinable, for example, by monitoring temperature changes at the tympanic membrane, in the rectum or in the bladder.

### EXAMPLES

Mild hypothermia is induced and maintained in healthy subjects for up to several hours. Table 1 summarizes data concerning the subjects for each phase of testing (Phases 1, 3 a, 4 and 5). Each volunteer is cooled using contact pads (Arctic Sun Energy Transfer Pads™, Medivance, Inc., Louisville, Colo., U.S.A.). Contact pads are applied to contact the subjects, and water at a controlled temperature is delivered to and circulated through the contact pads to effect the controlled cooling of each volunteer.

TABLE 1
Subject Characteristics

| Phase | Subject # | Age (yrs) | Sex | Weight (kg) | Height (cm) | BSA (M²) |
|---|---|---|---|---|---|---|
| 1 | 1 | 35 | M | 66 | 173 | 1.79 |
| | 2 | 40 | M | 91 | 183 | 2.13 |
| | 3 | 41 | F | 61 | 165 | 1.67 |
| | 4 | 36 | M | 91 | 183 | 2.13 |
| | 5 | 29 | F | 68 | 185 | 1.90 |
| | Mean ± SD | 36 ± 5 | | 75 ± 15 | 178 ± 13 | 1.92 ± .2 |
| 3a | 18 | 21 | F | 48 | 163 | 1.50 |
| | 19 | 22 | M | 98 | 193 | 2.29 |
| | 20 | 33 | F | 65 | 161 | 1.69 |
| | 22 | 25 | F | 48 | 164 | 1.50 |
| | 23 | 48 | F | 67 | 170 | 1.78 |
| | Mean ± SD | 30 ± 11 | | 65 ± 20 | 170 ± 13 | 1.75 ± .3 |
| 4 | 24 | 26 | F | 64 | 152 | 1.61 |
| | 25 | 39 | F | 74 | 157 | 1.75 |
| | 26 | 19 | M | 73 | 178 | 1.90 |
| | 27 | 26 | M | 64 | 165 | 1.70 |
| | Mean ± SD | 28 ± 8 | | 69 ± 6 | 163 ± 11 | 1.74 ± .1 |
| 5 | 29 | 37 | F | 51 | 168 | 1.57 |
| | 31 | 30 | M | 84 | 178 | 2.03 |
| | 32 | 35 | M | 71 | 178 | 1.88 |
| | 33 | 20 | F | 61 | 168 | 1.69 |
| | Mean ± SD | 31 ± 8 | | 67 ± 14 | 173 ± 6 | 1.79 ± .2 |

BSA = body surface area

Phase 1

Four contact pads are applied to the thighs and chest of each subject and each subject is rapidly cooled with an objective of reducing core body temperature to 34-35° C. Core body temperatures are measured at the tympanic membrane and the rectum. Mean skin-surface temperature is determined from the weighted average of calf, thigh, chest, and upper arm skin temperatures. Thermoregulatory vasoconstriction is evaluated using forearm minus fingertip skin-temperature gradients. Temperatures are measured using Mon-a-Therm™ themocouple probes connected to Mallinckrodt Model 6510 two-channel electronic thermometers having an accuracy near 0.1° C. (Mallinckrodt Anesthesia Products, St. Louis, Mo., U.S.A.). Temperatures are recorded before cooling is started (i.e., baseline) and subsequently at 15-minute intervals.

Each of subjects 1-5 is administered a single dose of acetomiophen (1000 mg) within 20 minutes prior to treatment and is administered a bolus of intravenous (IV) meperidine (Demerol®, 25-75 mg) within 5 minutes of the start of cooling. Subjects also receive initial doses of chlorpromazine (Thorazine®12.5-25 mg, IV). Active cooling is initiated and the inlet water temperature is controlled to achieve a target tympanic temperature between 34° C. and 35° C. Additional doses of meperidine and/or chloropromazine are administered to maintain comfort and to prevent shivering. Active cooling and maintenance of hypothermia continues for up to five hours, after which subjects are actively re-warmed to a tympanic temperature of 36° C.

The presence of shivering is noted by physical examination, electromyographic artifact on continuous electrocardiography (ECG), or by subject report. Overall thermal comfort is evaluated at 15-min intervals with a 100-mm-long visual analog scale (VAS) on which 0 mm defines the worst imaginable cold, 50 mm identifies thermal neutrality, and 100 mm indicates unbearable heat. A new, unmarked scale is used for each assessment. Heart rate and oxyhemoglobin saturation are monitored using ECG and pulse oximetry; arterial blood pressure is recorded oscillometrically at 15-minute intervals.

Phase 3a

Five contact pads are applied to the thighs, back and abdomen of each subjects, and actively cooled. Temperatures are measured in the same manner as in Phase 1 except that the rectal temperature probe is connected to a control module and the rectal temperature signal is used by the control module to adjust inlet water temperature via a feedback control algorithm to achieve a target core body temperature of 34.5° C.

A single oral dose of acetaminophen (1000 mg), is administered within 20 minutes prior to treatment. A bolus of IV meperidine (Demerol®, 50-100 mg) is given within 5 minutes of the start of active cooling. Additional doses of meperidine are administered to maintain comfort and to prevent shivering. Active cooling and maintenance of hypothermia continues for five hours. As in the Phase 1, the subjects are actively re-warmed to a tympanic temperature of 36° C. prior to termination of the experiment. Shivering, comfort, oxyhemoglobin saturation, and arterial blood pressure are recorded as in Phase 1.

Table 2 summarizes results for Phase 1 and Phase 3a. Results are expressed as mean SD. Five subjects are enrolled in Phase 1. Subject characteristics are summarized in Table 1. Mild hypothermia is attained in all subjects. The mean time to reach a tympanic temperature of 35° C. is 77+23 minutes which corresponds to a mean cooling rate of 1.5±0.6° C./hr. Details of the cooling responses of the individual subjects are presented in Table 2. The mean total dosage of meperidine is 280±155 mg. Only subjects #1 (37.5 mg) and #2 (12.5 mg) received chlorpromazine.

TABLE 2
Cooling Response of Subjects, Phases 1 & 3a

| | | | | Cooling | | Nausea or |
|---|---|---|---|---|---|---|
| | | | | rate | Total Meperidine | Vomiting |
| | | Tᵢₙᵢₜᵢₐₗ | Time to | to 35° C. | Dose | During |
| Phase | Subject # | (° C.) | 35° C. (min) | (° C./hr) | (mg) | Treatment |
| 1 | 1 | 36.8 | 41 | 2.6 | 75 | None |
| | 2 | 36.6 | 82 | 1.2 | 200 | None |
| | 3 | 36.9 | 81 | 1.4 | 275 | Pruritis |
| | 4 | 36.7 | 105 | 1.0 | 375 | None |
| | 5 | 37.0 | 78 | 1.5 | 475 | None |
| | Mean ± SD | 36.8 ± .2 | 77 ± 23 | 1.5 ± .6 | 280 ± 155 | |
| 3a | 18 | 37.0 | 64 | 1.9 | 400 | Nausea end |
| | 19 | 37.7 | 180 | .9 | 500 | None |
| | 20 | 36.9 | 95 | 1.2 | 350 | Nausea end |
| | 22 | 36.8 | 56 | 1.7 | 350 | Nausea early, |
| | | | | | | end |
| | 23 | 36.6 | 53 | 1.4 | 250 | Nausea, |
| | | | | | | vomiting mid |
| | Mean ± SD | 37.0 ± .4 | 90 ± 53 | 1.4 ± .4 | 370 ± 91 | |

T = tympanic membrane temperature

Six subjects are enrolled in the Phase 3a. One subject is withdrawn from the study prior to the initiation of cooling due to vasovagal syncope at the time of IV insertion. Subject characteristics of the remaining 5 subjects are presented in Table 1. The mean time to reach a tympanic temperature of 35° C. is 90±53 minutes corresponding to a mean cooling rate of 1.4±0.4° C./hr. Details of the cooling responses of the individual subjects are presented in Table 2. The mean total dosage of meperidine is 370±91 mg.

In all subjects, there is no statistically significant change in heart rate, diastolic blood pressure, or blood oxygenation compared with baseline. Systolic blood pressure is significantly elevated compared with baseline only for the 180 min timepoint (140±20 vs 122±13 mm Hg; p=0.042). The mean total meperidine dosage in Phase 3a is 90 mg higher than in Phase 1 (370 mg vs 280 mg; p=0.28, t-test). Comfort is statistically significantly lower than baseline for all timepoints during active cooling (except T=135 min) although no subject requested that the study be terminated.

In Phases 1 and 3a, meperidine is used to suppress shivering and to maintain comfort. Although no respiratory compromise is observed, nausea is observed in 30% of subjects. All cases of nausea occurred in Phase 3a making the trend toward a higher total meperidine dosage in Phase 3a noteworthy.

Phase 4

Phase 4 proceeds as described for Phase 3a, except as noted. In addition to meperidine (Demerol®), subjects are also given an oral dose of 30-60 mg buspirone. Subject characteristics are summarized in Table 1. Results are summarized in Table 3.

Five subjects are enrolled in Phase 4. One subject is withdrawn due to sustained nausea and vomiting throughout the treatment period. Details of the cooling responses of the individual subjects are presented in Table 3. As seen in Table 3, the use of buspirone did not reduce the incidence of nausea in those four subjects.

TABLE 3
Cooling Response of Subjects, Phase 4

| | | | | Cooling | | Nausea or |
|---|---|---|---|---|---|---|
| | | | Time | rate | Total Meperidine | Vomiting |
| | | Tᵢₙᵢₜᵢₐₗ | to 35° C. | to 35° C. | Dose | During |
| Phase | Subject # | (° C.) | (min) | (° C./hr) | (mg) | Treatment |
| 4 | 24 | 36.9 | 63 | 1.9 | 250 | Nausea mid |
| | 25 | 37.3 | 158 | 0.8 | 400 | Nausea end |
| | 26 | 37.0 | 105 | 1.1 | 250 | Nausea early, |
| | | | | | | Vomiting end |
| | 27 | 36.8 | 95 | 1.1 | 250 | None |
| | Mean ± SD | 37.0 ± .2 | 105 ± 39 | 1.2 ± .5 | 288 ± 75 | |

T = tympanic membrane temperature

Phase 5

Phase 5 proceeds as described for Phase 3a, except as noted. The temperature of water circulated to the contact pads is controlled to achieve a target core body temperature of 34.5° C. In addition to meperidine (Demerol®), subjects are also given an IV dose of 8 mg ondansetron (hydrochloride salt, Glaxo Wellcome). Subject characteristics are summarized in Table 1. Results are summarized in Table 4.

Five subjects are enrolled in Phase 5. One subject is withdrawn due to a mild allergic reaction to meperidine, which caused a facial rash. Mild hypothermia is attained in all subjects the mean time to reach a tympanic temperature of 35.0° C. is 72±15 minutes, corresponding to a cooling rate of 1.6° C./hr. Details of the cooling responses of the individual subjects are presented in Table 4. The mean total dosage of meperidine is 306±55 mg.

Subject #29 feels very warm at the end of the treatment, and becomes nauseated and vomits immediately after the treatment. The nausea and vomiting is believed to be due to a fast rate of warming of the subject. The rate of warming is reduced for subsequent subjects, who do not become nauseated and do not vomit during the treatment. Thus, the use of ondansetron reduces the incidence of nausea and vomiting, relative to Phases 3a and 4. In addition, there is a trend toward less meperidine administration in Phase 5 relative to Phase 3a (p=0.26, t-test). Furthermore, the mean comfort score for the subjects is higher in Phase 5 than in Phases 1, 3a and 4.

TABLE 4
Cooling Response of Subjects, Phase 5

| | | | | Cooling | Total | |
|---|---|---|---|---|---|---|
| | | | Time to | rate | Meperidine | |
| | | Tᵢₙᵢₜᵢₐₗ | 35° C. | to 35° C. | Dose | Nausea or Vomiting |
| Phase | Subject # | (° C.) | (min) | (° C./hr) | (mg) | During Treatment |
| 5 | 29 | 36.9 | 60 | 1.9 | 375 | Nausea and vomiting |
| | | | | | | immediately post |
| | 31 | 37.0 | 80 | 1.5 | 275 | None |
| | 32 | 36.4 | 58 | 1.4 | 250 | None |
| | 33 | 37.3 | 88 | 1.6 | 325 | Nausea post |
| | Mean ± SD | 36.9 ± .4 | 72 ± 15 | 1.6 ± .2 | 306 ± 55 | |

T = tympanic membrane temperature

Moreover, during a subsequent test conducted in a manner similar to that described for Phase 5, a subject was administered an initial dose of 8 mg of the ondansetron followed by a supplemental dose of 8 mg of the ondansetron administered 3.5 hours later. The subject did not experience nausea.

The foregoing discussion of the invention has been presented for purposes of illustration and description. The foregoing is not intended to limit the invention to only the form or forms specifically disclosed herein. Although the description of the invention has included description of one or more possible implementations and certain variations and modifications, other variations and modifications are within the scope of the invention, e.g., as may be within the skill and knowledge of those in the art after understanding the present disclosure. It is intended to obtain rights which include alternative embodiments to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter. Furthermore, any feature described with respect to any disclosed implementation may be combined in any combination with one or more features of any other implementation or implementations. Moreover, the invention specifically includes use of the anti-shivering drug and the anti-emetic drug in any implementation disclosed herein, the manufacture of the anti-emetic drug and/or the anti-shivering drug for use to manufacture a medicament for use in any of the disclosed herein, and any other use, manipulation or processing of the anti-emetic drug and/or the anti-shivering drug in preparation for use in any implementation disclosed herein.

The terms “comprise”, “include”, “have” and “contain”, and variations of those terms, as may be used in relation to the presence any feature described or claimed, are intended to indicate only that a particular feature is present to an extent as specified, and are not intended to limit the presence of that feature beyond the extent specified or the presence of other features.

What is claimed is:

1. A method for lowering the body temperature of a human, the method comprising:
transferring heat from the body of the human;
administering to the human an anti-emetic drug in an effective quantity for treatment to suppress nausea during the transferring; and
administering to the human an anti-shivering drug in effective quantity for treatment to suppress shivering during the transferring.

2. The method of claim 1, wherein during the administering the anti-emetic drug, a dose of the anti-emetic drug is administered to the human prior to commencement of the transferring.

3. The method of claim 1, wherein during the administering the anti-emetic drug, a dose of the anti-emetic drug is administered to the human prior to commencement of the administering of the anti-shivering drug.

4. The method of claim 1, wherein the administering the anti-shivering drug comprises a multi-dose administration regimen;
the multiple-dose administration regimen comprising a first dose of the anti-shivering drug administered to the human prior to commencement of or during the transferring;
the multiple-dose administration regiment comprising a second dose of the anti-shivering drug administered to the human during the transferring and after administration of the first dose.

5. The method of claim 4, wherein the administering the anti-emetic drug comprises administering a dose of the anti-emetic drug prior to the first dose of the anti-shivering drug.

6. The method of claim 1, wherein the anti-shivering drug comprises an analgesic.

7. The method of claim 1, wherein the anti-shivering drug comprises an opioid analgesic.

8. The method of claim 7, wherein the opioid analgesic is selected from the group consisting of alfentanil, morphine, fentanyl, meperidine, naloxone, nalbuphine and combinations of any of the foregoing.

9. The method of claim 1, wherein the anti-shivering drug comprises meperidine.

10. The method of claim 1, wherein the anti-emetic drug comprises a serotonin antagonist.

11. The method of claim 10, wherein the anti-emetic drug comprises a 5-HT₃ antagonist.

12. The method of claim 11, wherein the anti-emetic drug is selected from the group consisting of ondansetron, granisetron, tropisetron and combinations of any of the foregoing.

13. The method of claim 1, wherein the anti-emetic drug comprises ondansetron.

14. The method of claim 1, wherein the anti-shivering drug comprises meperidine and the anti-emetic drug comprises ondansetron.

15. The method of claim 1, wherein the transferring comprises transfer of heat from the body of the human into a heat transfer fluid brought into heat transfer communication with the human body.

16. The method of claim 15, wherein the transferring comprises flowing the heat transfer fluid through a heat transfer device in heat transfer communication with an exterior dermal surface, whereby heat is transferred from the human across the dermal surface and into the heat transfer fluid flowing through the heat transfer device.

17. The method of claim 16, wherein the heat transfer device comprises a contact cooling pad and the heat transfer fluid is a liquid.

18. The method of claim 17, wherein the liquid comprises water.

19. The method of claim 17, wherein the contact cooling pad comprises an adherent surface in contact with the dermal surface during the transferring.

20. The method of claim 19, wherein the adherent surface comprises a thermally conductive hydrogel layer.

21. The method of claim 15, wherein during the transferring, the heat transfer fluid is introduced into a heat transfer device located within the vasculature of the human.

22. The method of claim 1, wherein the transferring comprises reducing the core temperature of the human by at least 1° C.

23. The method of claim 1, wherein the transferring comprises reducing the core temperature of the human in a range of from 1° C. to 5° C.

24. The method of claim 1, wherein the anti-emetic drug reduces the quantity of the anti-shivering drug required during the administering the anti-shivering drug for the treatment to suppress shivering during the transferring.

25. The method of claim 1, wherein during the transferring comprises inducing hypothermia in the human.

26. The method of claim 25, wherein the human is suffering a condition involving reduced blood supply.

27. The method of claim 26, wherein the condition comprises at least one of stroke, myocardial infarction and cardiac arrest.

28. A method for lowering the body temperature of a human, the method comprising:
transferring heat from the body of the human;
administering to the human prior to or during the transferring an opioid analgesic; and
administering to the human prior to or during the transferring a 5-HT₃ antagonist;
the opioid analgesic and the 5-HT₃ antagonist being administered in effective quantities for treatment to manage shivering and nausea during the transferring.

29. The method of claim 28, wherein the transferring heat from the body of the human comprises lowering the core temperature of the human by at least 0.5° C.

30. The method of claim 29, wherein the transferring heat from the body of the human to a heat exchange fluid contained within a heat exchange device in heat transfer communication with the body of the human.

31. The method of claim 30, wherein during the transferring heat from the body of the human, the heat exchange device is in heat transfer communication with a dermal surface of the human and heat is transferred across the dermal surface and to the heat exchange fluid.

32. The method of claim 31, wherein the heat exchange device comprises a contact cooling pad and the heat exchange fluid is a liquid flowing through the contact cooling-pad.

33. The method of claim 30, wherein the administering the anti-emetic drug comprises administering a dose of the anti-emetic drug to the human prior to commencement of the transferring heat from the body of the human.

34. The method of claim 33, wherein the administering the anti-shivering drug comprises a multiple dose administration regimen comprising at least two separate doses of the anti-shivering drug administered to the human after the administering the dose of the anti-emetic drug to the human.

35. The method of claim 33, wherein the 5-HT3 antagonist comprises ondansetron.

36. The method of claim 35, wherein the opioid analgesic comprises meperidine.

37. A method for suppressing shivering during active cooling of a human body, comprising administering a 5-HT₃ antagonist to the human prior to or during the active cooling.

38. The method of claim 37, wherein the active cooling comprises inducing.
